(19) United States
(12) Patent Application Publication
(10) Pub. No.: US 2024/0207256 A1
(43) Pub. Date: Jun. 27, 2024

(54) CERTAIN CHEMICAL ENTITIES, COMPOSITIONS, AND METHODS

(71) Applicant: NeuPharma, Inc, San Mateo, CA (US)

(72) Inventor: Xiangping QIAN, Foster City, CA (US)

(21) Appl. No.: 18/560,312

(22) PCT Filed: May 11, 2022
(86) PCT No.: PCT/US2022/028833

§ 371 Date: Nov. 10, 2023

Related U.S. Application Data
(60) Provisional application No. 63/187,823, filed on May 12, 2021.

(51) Int. Cl. A61K 31/496; A61K 9/16; A61K 47/10; A61P 31/00

(52) U.S. Cl. CPC A61K 31/496; A61K 9/1617; A61K 47/10; A61P 31/00

(57) ABSTRACT

Described herein are chemoembolization compositions comprising an anti-cancer drug, methods of making, and their use in treating cancer, such as liver cancer.

### CROSS-REFERENCE

This application claims the benefit of U.S. Provisional Application No. 63/187,823 filed May 12, 2021, which is hereby incorporated by reference in its entirety.

### BACKGROUND OF THE INVENTION

Cancer can be viewed as a breakdown in the communication between tumor cells and their environment, including their normal neighboring cells. Signals, both growth-stimulatory and growth-inhibitory, are routinely exchanged between cells within a tissue. Normally, cells do not divide in the absence of stimulatory signals, and likewise, will cease dividing in the presence of inhibitory signals. In a cancerous, or neoplastic state, a cell acquires the ability to “override” these signals and to proliferate under conditions in which normal cells would not grow.

Cardiotonic steroids like digoxin and digitoxin are a class of naturally derived compounds that bind to and inhibit Na⁺/K⁺-ATPase (sodium pump). Members of this family have been used for the treatment of heart failure and arrhythmia for many years. Recent findings have revealed that these compounds may be involved in the regulation of several important cellular processes. Several cardiotonic steroids such as digitoxin and oleandrin have shown inhibitory effect on the growth of human tumor cells.

### SUMMARY OF THE INVENTION

In one aspect, the disclosure provides a chemoembolization composition comprising (3S,5R,8R,9S,10S,13R,14S,17R)-14-hydroxy-10,13-dimethyl-17-(2-oxo-2H-pyran-5-yl)hexadecahydro-1H-cyclopenta[α]phenanthren-3-yl piperazine-1-carboxylate (Compound A):

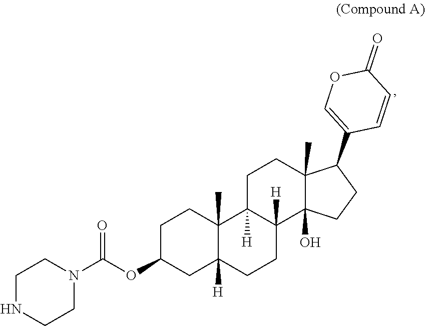

or a pharmaceutically acceptable salt thereof, loaded in drug-eluting beads.

In some embodiments, the drug-eluting beads are anionic drug-eluting beads. In some embodiments, the drug-eluting beads comprise modified polyvinyl alcohol (PVA) hydrogel beads. In some embodiments, the drug-eluting beads comprise sulfonate-modified polyvinyl alcohol hydrogel beads or carboxyl-modified polyvinyl alcohol acrylate beads. In some embodiments, the drug-eluting beads comprise DC Bead®, HepaSphere®, or CalliSpheres® drug-eluting beads.

In some embodiments, the drug-eluting beads have an average diameter of about 70 μm to about 700 μm. In some embodiments, the drug-eluting beads have an average diameter of about 70 μm to about 150 μm, of about 100 μm to about 300 μm, of about 300 μm to about 500 μm, or of about 500 μm to about 700 μm. In some embodiments, the drug-eluting beads have an average diameter of about 100 μm to about 300 μm.

In some embodiments, the compound is (3S,5R,8R,9S,10S,13R,14S,17R)-14-hydroxy-10,13-dimethyl-17-(2-oxo-2H-pyran-5-yl)hexadecahydro-1H-cyclopenta[α]phenanthren-3-yl piperazine-1-carboxylate (Compound A).

In some embodiments, the chemoembolization composition comprises from about 0.5 mg to about 5 mg of Compound A per 1 g of drug-eluting beads. In some embodiments, the chemoembolization composition comprises from about 1 mg to about 2 mg of Compound A per 1 g of drug-eluting beads. In some embodiments, the chemoembolization composition comprises from about 1.4 mg to about 1.6 mg of Compound A per 1 g of drug-eluting beads.

In another aspect, the disclosure provides the chemoembolization composition provided herein for use in a method for treating a solid tumor cancer.

In some embodiments, the solid tumor cancer comprises a malignant hypervascularised tumor. In some embodiments, the malignant hypervascularised tumor is selected from the group comprising hepatoma, hepatocellular carcinoma (HCC), liver metastasis, cholangiomas, neuroendocrine tumors, GIST liver metastasis, and renal cancer.

In another aspect, the disclosure provides an aqueous chemoembolization composition comprising (3S,5R,8R,9S,10S,13R,14S,17R)-14-hydroxy-10,13-dimethyl-17-(2-oxo-2H-pyran-5-yl)hexadecahydro-1H-cyclopenta[α]phenanthren-3-yl piperazine-1-carboxylate (Compound A):

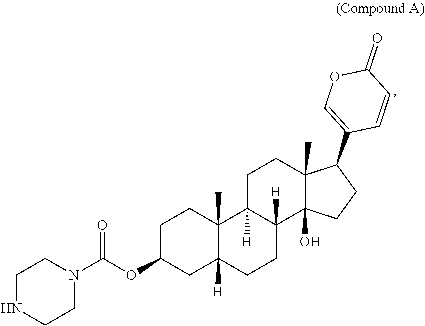

or a pharmaceutically acceptable salt thereof, loaded in drug-eluting beads.

In some embodiments, the aqueous chemoembolization composition further comprises a buffer. In some embodiments, the buffer comprises phosphoric acid, citric acid, acetic acid, histidine, lactic acid, tromethamine, gluconic acid, aspartic acid, glutamic acid, tartaric acid, succinic acid, malic acid, fumaric acid, α-ketoglutaric acid, sodium hydroxide, sodium phosphate, sodium citrate, sodium acetate, potassium hydroxide, potassium phosphate, potassium citrate, potassium acetate, or a combination thereof. In some embodiments, the buffer comprises sodium acetate, acetic acid, or a combination thereof.

In some embodiments, the buffer has a concentration from about 10 mM to about 500 mM. In some embodiments, the buffer has a concentration of about 100 mM.

In some embodiments, the pH of the aqueous chemoembolization composition is from about 3.5 to about 7.5. In some embodiments, the pH of the aqueous chemoembolization composition is from about 4.5 to about 5.5. In some embodiments, the pH of the aqueous chemoembolization composition is about 5.0.

In some embodiments, the drug-eluting beads are anionic drug-eluting beads. In some embodiments, the drug-eluting beads comprise modified polyvinyl alcohol (PVA) hydrogel beads. In some embodiments, the drug-eluting beads comprise sulfonate-modified polyvinyl alcohol hydrogel beads or carboxyl-modified polyvinyl alcohol acrylate beads. In some embodiments, the drug-eluting beads comprise DC Bead®, HepaSphere®, or CalliSpheres® drug-eluting beads.

In some embodiments, the drug-eluting beads have an average diameter of about 70 μm to about 700 μm. In some embodiments, the drug-eluting beads have an average diameter of about 70 μm to about 150 μm, of about 100 μm to about 300 μm, of about 300 μm to about 500 μm, or of about 500 μm to about 700 μm. In some embodiments, the drug-eluting beads have an average diameter of about 100 μm to about 300 μm.

In some embodiments, the concentration of the drug-eluting beads is from about 0.1 g/mL to about 1 g/mL. In some embodiments, the concentration of the drug-eluting beads is from about 0.1 g/mL to about 0.5 g/mL. In some embodiments, the concentration of the drug-eluting beads is about 0.25 g/mL.

In some embodiments, the compound is (3S,5R,8R,9S,10S,13R,14S,17R)-14-hydroxy-10,13-dimethyl-17-(2-oxo-2H-pyran-5-yl)hexadecahydro-1H-cyclopenta[α]phenanthren-3-yl piperazine-1-carboxylate (Compound A).

In some embodiments, the aqueous chemoembolization composition comprises from about 0.5 mg to about 5 mg of Compound A per 1 g of drug-eluting beads. In some embodiments, the aqueous chemoembolization composition comprises from about 1 mg to about 2 mg of Compound A per 1 g of drug-eluting beads. In some embodiments, the aqueous chemoembolization composition comprises from about 1.4 mg to about 1.6 mg of Compound A per 1 g of drug-eluting beads.

In another aspect, the disclosure provides the aqueous chemoembolization composition provided herein for use in a method for treating a solid tumor cancer.

In some embodiments, the solid tumor cancer comprises a malignant hypervascularised tumor. In some embodiments, the malignant hypervascularised tumor is selected from the group comprising hepatoma, hepatocellular carcinoma (HCC), liver metastasis, cholangiomas, neuroendocrine tumors, GIST liver metastasis, and renal cancer.

In another aspect, the disclosure provides a method of treating a solid tumor cancer in a subject, the method comprising administering to the subject in need thereof a chemoembolization composition comprising a therapeutically effective amount of (3S,5R,8R,9S,10S,13R,14S,17R)-14-hydroxy-10,13-dimethyl-17-(2-oxo-2H-pyran-5-yl)hexadecahydro-1H-cyclopenta[α]phenanthren-3-yl piperazine-1-carboxylate (Compound A):

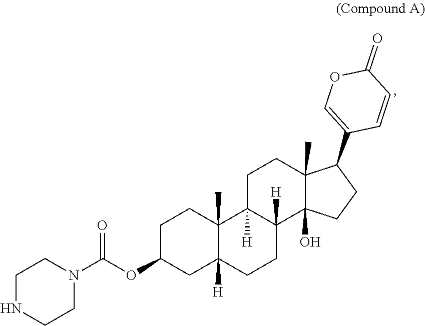

or a pharmaceutically acceptable salt thereof, loaded in drug-eluting beads.

In some embodiments, the solid tumor cancer comprises a malignant hypervascularised tumor. In some embodiments, the malignant hypervascularised tumor is selected from the group comprising hepatoma, hepatocellular carcinoma (HCC), liver metastasis, cholangiomas, neuroendocrine tumors, GIST liver metastasis, and renal cancer. In some embodiments, the solid tumor cancer is unresectable

In another aspect, the disclosure provides a method of treating a hepatocellular carcinoma (HCC) in a subject, the method comprising administering to the subject in need thereof a chemoembolization composition comprising a therapeutically effective amount of (3S,5R,8R,9S,10S,13R,14S,17R)-14-hydroxy-10,13-dimethyl-17-(2-oxo-2H-pyran-5-yl)hexadecahydro-1H-cyclopenta[α]phenanthren-3-yl piperazine-1-carboxylate (Compound A):

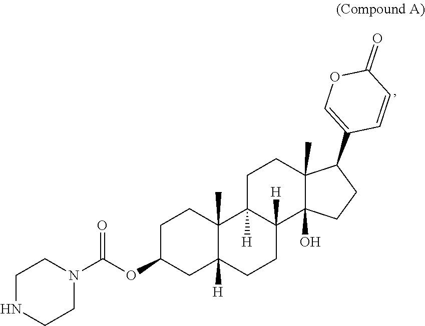

or a pharmaceutically acceptable salt thereof, loaded in drug-eluting beads.

In some embodiments, the HCC is unresectable.

In some embodiments, the chemoembolization composition is administered directly to the hepatic artery in the liver of the subject.

In some embodiments, the chemoembolization composition comprises from about 1.4 mg to about 1.6 mg of Compound A and about 1 g of drug-eluting beads.

In some embodiments, the chemoembolization composition is the chemoembolization composition provided herein.

In some embodiments, the chemoembolization composition is the aqueous chemoembolization composition provided herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

Drug eluting beads (DEBs) have emerged as a means of delivering chemotherapy in a targeted and controllable manner while lowering systemic toxicity and sustaining local activity. The use of DEBs in embolization therapies, such as transarterial chemoembolization (TACE), combines the therapeutic effects of peripheral arterial occlusion with the local administration of chemotherapeutic agents. Because 95% of the blood supplied to liver cancer is supplied via the hepatic artery, while more than 75% of the blood supplied to the surrounding healthy tissue is supplied via the hepatic portal vein, DEB-TACE is ideally suited for the targeted treatment of liver cancer.

A significant proportion of HCC patients present with, or progress to, intermediate stage disease, and these patients are typically treated with transarterial chemoembolization (TACE) or transarterial embolization (TAE). However, since TACE/TAE is generally a palliative therapy, it provides a potential backbone for the addition of effective systemic therapies with the aim of improving survival outcomes. Compound A is a potent Na+/K+-ATPase inhibitor with an IC50 of about 20 nM against Na+/K+-ATPase. Through its primary target of Na+/K+-ATPase, Compound A exerts its anti-cancer activity through multiple signal transduction pathways including cell-cycle arrest, apoptosis and autophagic cell death.

### Compound A

As used herein, Compound A refers to (3S,5R,8R,9S,10S,13R,14S,17R)-14-hydroxy-10,13-dimethyl-17-(2-oxo-2H-pyran-5-yl)hexadecahydro-1H-cyclopenta[α]phenanthren-3-yl piperazine-1-carboxylate, which has the chemical structure shown below:

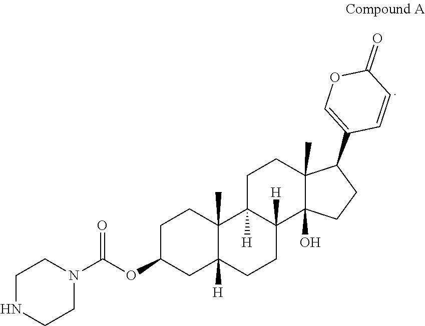

Compound A has been prepared previously (see, WO 2011/085641, U.S. Pat. Nos. 8,334,376, 8,993,550, 9,399,659, 9,814,735, 10,179,141, 10,471,078, and U.S. patent application Ser. No. 16/584,263).

### I. Additional Definitions

As used herein, “active agent” is used to indicate a chemical entity which has biological activity. In certain embodiments, an “active agent” is a compound having pharmaceutical utility. For example an active agent may be an anti-cancer therapeutic.

As used herein, “modulation” refers to a change in activity as a direct or indirect response to the presence of a chemical entity as described herein, relative to the activity of in the absence of the chemical entity. The change may be an increase in activity or a decrease in activity, and may be due to the direct interaction of the compound with the target or due to the interaction of the compound with one or more other factors that in turn affect the target's activity. For example, the presence of the chemical entity may, for example, increase or decrease the target activity by directly binding to the target, by causing (directly or indirectly) another factor to increase or decrease the target activity, or by (directly or indirectly) increasing or decreasing the amount of target present in the cell or organism.

As used herein, “therapeutically effective amount” of a chemical entity described herein refers to an amount effective, when administered to a human or non-human subject, to provide a therapeutic benefit such as amelioration of symptoms, slowing of disease progression, or prevention of disease.

“Treating” or “treatment” encompasses administration of Compound A, or a pharmaceutically acceptable salt thereof, to a mammalian subject, particularly a human subject, in need of such an administration and includes (i) arresting the development of clinical symptoms of the disease, such as cancer, (ii) bringing about a regression in the clinical symptoms of the disease, such as cancer, and/or (iii) prophylactic treatment for preventing the onset of the disease, such as cancer.

As used herein, a “pharmaceutically acceptable” component is one that is suitable for use with humans and/or animals without undue adverse side effects (such as toxicity, irritation, and allergic response) commensurate with a reasonable benefit/risk ratio.

“Pharmaceutically acceptable salts” include, but are not limited to salts with inorganic acids, such as hydrochlorate, carbonate, phosphate, hydrogenphosphate, diphosphate, hydrobromate, sulfate, sulfinate, nitrate, and like salts; as well as salts with an organic acid, such as malate, malonate, maleate, fumarate, tartrate, succinate, citrate, acetate, lactate, gluconate, methanesulfonate, Tris (hydroxymethyl-aminomethane), p-toluenesulfonate, propionate, 2-hydroxyethylsulfonate, benzoate, salicylate, stearate, oxalate, pamoate, and alkanoate such as acetate, HOOC—(CH₂)ₙ—COOH where n is 0-4, and like salts. Other salts include sulfate, methanesulfonate, bromide, trifluoroacetate, picrate, sorbate, benzilate, salicylate, nitrate, phthalate or morpholine. Pharmaceutically acceptable cations include, but are not limited to sodium, potassium, calcium, aluminum, lithium, and ammonium.

In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts.

As used herein, “subject” refers to a mammal that has been or will be the object of treatment, observation or experiment. The methods described herein can be useful in both human therapy and veterinary applications. In some embodiments, the subject is a human.

The term “mammal” is intended to have its standard meaning, and encompasses humans, dogs, cats, sheep, and cows, for example.

“Prodrugs” described herein include any compound that becomes Compound A when administered to a subject, e.g., upon metabolic processing of the prodrug. Similarly, “pharmaceutically acceptable salts” includes “prodrugs” of pharmaceutically acceptable salts. Examples of prodrugs include derivatives of functional groups, such as a carboxylic acid group, in Compound A. Exemplary prodrugs of a carboxylic acid group include, but are not limited to, carboxylic acid esters such as alkyl esters, hydroxyalkyl esters, arylalkyl esters, and aryloxyalkyl esters. Other exemplary prodrugs include lower alkyl esters such as ethyl ester, acyloxyalkyl esters such as pivaloyloxymethyl (POM), glycosides, and ascorbic acid derivatives. Other exemplary prodrugs include amides of carboxylic acids. A discussion of prodrugs is provided in T. Higuchi and V. Stella, Pro-drugs as Novel Delivery Systems, Vol. 14 of the A.C.S. Symposium Series, in Edward B. Roche, ed., Bioreversible Carriers in Drug Design, American Pharmaceutical Association and Pergamon Press, 1987, and in Design of Prodrugs, ed. H. Bundgaard, Elsevier, 1985.

The compounds disclosed herein can be used in different enriched isotopic forms, e.g., enriched in the content of ²H, ³H, ¹¹C, ¹³C and/or ¹⁴C. In one particular embodiment, the compound is deuterated at least one position. Such deuterated forms can be made by the procedure described in U.S. Pat. Nos. 5,846,514 and 6,334,997. As described in U.S. Pat. Nos. 5,846,514 and 6,334,997, deuteration can improve the efficacy and increase the duration of action of drugs.

Deuterium substituted compounds can be synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6 (10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64 (1-2), 9-32.

A “solvate” is formed by the interaction of a solvent and a compound. The term “compound” is intended to include solvates of compounds. Similarly, “pharmaceutically acceptable salts” includes solvates of pharmaceutically acceptable salts. Suitable solvates are pharmaceutically acceptable solvates, such as hydrates, including monohydrates and hemi-hydrates. Also included are solvates formed with the one or more crystallization solvents.

Pharmaceutically acceptable forms of the compounds recited herein include pharmaceutically acceptable salts, chelates, non-covalent complexes, prodrugs, and mixtures thereof.

A “chelate” is formed by the coordination of a compound to a metal ion at two (or more) points. The term “compound” is intended to include chelates of compounds. Similarly, “pharmaceutically acceptable salts” includes chelates of pharmaceutically acceptable salts.

A “non-covalent complex” is formed by the interaction of a compound and another molecule wherein a covalent bond is not formed between the compound and the molecule. For example, complexation can occur through van der Waals interactions, hydrogen bonding, and electrostatic interactions (also called ionic bonding). Such non-covalent complexes are included in the term “compound”. Similarly, pharmaceutically acceptable salts include “non-covalent complexes” of pharmaceutically acceptable salts.

When ranges are used herein for physical properties, such as molecular weight, or chemical properties, such as chemical formulae, all combinations and sub combinations of ranges and specific embodiments therein are intended to be included.

The term “about” when referring to a number or a numerical range means that the number or numerical range referred to is an approximation within experimental variability (or within statistical experimental error), and thus the number or numerical range may vary from, for example, between 1% and 15% of the stated number or numerical range. In some instances of numerical ranges, “about” means+10%.

As used herein, “significant” refers to any detectable change that is statistically significant in a standard parametric test of statistical significance such as Student's T-test, where p<0.05.

As used herein, “cancer” refers to all types of cancer or neoplasm or malignant tumors found in mammals, including carcinomas and sarcomas. Examples of cancer are cancer of the brain, breast, cervix, colon, head & neck, kidney, lung, non-small cell lung, melanoma, mesothelioma, ovary, sarcoma, stomach, uterus and Medulloblastoma.

### II. Chemoembolization Compositions of Compound A

In one aspect, the disclosure provides a chemoembolization composition comprising (3S,5R,8R,9S,10S,13R,14S,17R)-14-hydroxy-10,13-dimethyl-17-(2-oxo-2H-pyran-5-yl)hexadecahydro-1H-cyclopenta[α]phenanthren-3-yl piperazine-1-carboxylate (Compound A):

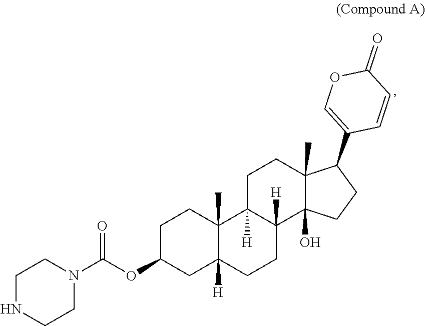

or a pharmaceutically acceptable salt thereof, loaded in drug-eluting beads.

In some embodiments, the drug-eluting beads are anionic drug-eluting beads. In some embodiments, the drug-eluting beads comprise modified polyvinyl alcohol (PVA) hydrogel beads. In some embodiments, the drug-eluting beads comprise sulfonate-modified polyvinyl alcohol hydrogel beads or carboxyl-modified polyvinyl alcohol acrylate beads. In some embodiments, the drug-eluting beads comprise sulfonate-modified polyvinyl alcohol hydrogel beads. In some embodiments, the drug-eluting beads comprise carboxyl-modified polyvinyl alcohol acrylate beads. In some embodiments, the drug-eluting beads comprise carboxyl-modified polyvinyl alcohol-co-sodium acrylate beads. In some embodiments, the drug-eluting beads are water-insoluble. In some embodiments, the drug-eluting beads are water-swellable. In some embodiments, the drug-eluting beads are imageable. In some embodiments, the drug-eluting beads are radiopaque. In some embodiments, the drug-eluting beads comprise DC Bead®, HepaSphere®, or CalliSpheres® drug-eluting beads. In some embodiments, the drug-eluting beads comprise DC Bead® drug-eluting beads. In some embodiments, the drug-eluting beads comprise HepaSphere® drug-eluting beads. In some embodiments, the drug-eluting beads comprise CalliSpheres® drug-eluting beads.

In some embodiments, the drug-eluting beads have an average diameter of about 30 μm to about 1200 μm. In some embodiments, the drug-eluting beads have an average diameter of about 70 μm to about 700 μm. In some embodiments, the drug-eluting beads have an average diameter of about 70 μm to about 150 μm, of about 100 μm to about 300 μm, of about 300 μm to about 500 μm, or of about 500 μm to about 700 μm. In some embodiments, the drug-eluting beads have an average diameter of about 30 μm to about 60 μm. In some embodiments, the drug-eluting beads have an average diameter of about 50 μm to about 100 μm. In some embodiments, the drug-eluting beads have an average diameter of about 70 μm to about 150 μm. In some embodiments, the drug-eluting beads have an average diameter of about 100 μm to about 200 μm. In some embodiments, the drug-eluting beads have an average diameter of about 150 μm to about 200 μm. In some embodiments, the drug-eluting beads have an average diameter of about 100 μm to about 300 μm. In some embodiments, the drug-eluting beads have an average diameter of about 300 μm to about 500 μm. In some embodiments, the drug-eluting beads have an average diameter of about 500 μm to about 700 μm. In some embodiments, the drug-eluting beads have an average diameter of about 700 μm to about 900 μm. In some embodiments, the drug-eluting beads have an average diameter of about 900 μm to about 1200 μm.

In some embodiments, the compound is (3S,5R,8R,9S,10S,13R,14S,17R)-14-hydroxy-10,13-dimethyl-17-(2-oxo-2H-pyran-5-yl)hexadecahydro-1H-cyclopenta[α]phenanthren-3-yl piperazine-1-carboxylate (Compound A). In some embodiments, Compound A is provided as a free base. In some embodiments, Compound A is provided as an acid addition salt.

In some embodiments, the chemoembolization composition comprises from about 0.5 mg to about 5 mg of Compound A per 1 g of drug-eluting beads. In some embodiments, the chemoembolization composition comprises from about 1 mg to about 2 mg of Compound A per 1 g of drug-eluting beads. In some embodiments, the chemoembolization composition comprises from about 1.4 mg to about 1.6 mg of Compound A per 1 g of drug-eluting beads. In some embodiments, the chemoembolization composition comprises about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, or about 2.0 mg of Compound A per 1 g of drug-eluting beads. In some embodiments, the chemoembolization composition comprises about 1.4 mg of Compound A per 1 g of drug-eluting beads. In some embodiments, the chemoembolization composition comprises about 1.5 mg of Compound A per 1 g of drug-eluting beads. In some embodiments, the chemoembolization composition comprises about 1.6 mg of Compound A per 1 g of drug-eluting beads.

In some embodiments, the disclosure provides a dried chemoembolization composition comprising Compound A. In other embodiments, the disclosure provides a liquid chemoembolization composition comprising Compound A. In some embodiments, the liquid chemoembolization composition is a solution. In some embodiments, the liquid chemoembolization composition is a suspension. In some embodiments, the liquid chemoembolization composition is an aqueous chemoembolization composition.

In another aspect, the disclosure provides an aqueous chemoembolization composition comprising (3S,5R,8R,9S,10S,13R,14S,17R)-14-hydroxy-10,13-dimethyl-17-(2-oxo-2H-pyran-5-yl)hexadecahydro-1H-cyclopenta[α]phenanthren-3-yl piperazine-1-carboxylate (Compound A):

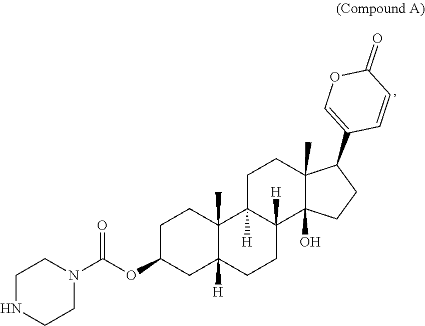

or a pharmaceutically acceptable salt thereof, loaded in drug-eluting beads.

In some embodiments, the aqueous chemoembolization composition further comprises a buffer. In some embodiments, the buffer comprises phosphoric acid, citric acid, acetic acid, histidine, lactic acid, tromethamine, gluconic acid, aspartic acid, glutamic acid, tartaric acid, succinic acid, malic acid, fumaric acid, α-ketoglutaric acid, sodium hydroxide, sodium phosphate, sodium citrate, sodium acetate, potassium hydroxide, potassium phosphate, potassium citrate, potassium acetate, or a combination thereof. In some embodiments, the buffer comprises sodium acetate, acetic acid, or a combination thereof.

In some embodiments, the buffer has a concentration from about 10 mM to about 500 mM. In some embodiments, the buffer has a concentration from about 50 mM to about 200 mM. In some embodiments, the buffer has a concentration of about 10 mM, about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 250 mM, about 300 mM, about 350 mM, about 400 mM, about 450 mM, or about 500 mM. In some embodiments, the buffer has a concentration of about 100 mM.

In some embodiments, the pH of the aqueous chemoembolization composition is from about 3.5 to about 7.5. In some embodiments, the pH of the aqueous chemoembolization composition is from about 4.5 to about 5.5. In some embodiments, the pH of the aqueous chemoembolization composition is about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5. In some embodiments, the pH of the aqueous chemoembolization composition is about 5.0.

In some embodiments, the drug-eluting beads are anionic drug-eluting beads. In some embodiments, the drug-eluting beads comprise modified polyvinyl alcohol (PVA) hydrogel beads. In some embodiments, the drug-eluting beads comprise sulfonate-modified polyvinyl alcohol hydrogel beads or carboxyl-modified polyvinyl alcohol acrylate beads. In some embodiments, the drug-eluting beads comprise sulfonate-modified polyvinyl alcohol hydrogel beads. In some embodiments, the drug-eluting beads comprise carboxyl-modified polyvinyl alcohol acrylate beads. In some embodiments, the drug-eluting beads comprise carboxyl-modified polyvinyl alcohol-co-sodium acrylate beads. In some embodiments, the drug-eluting beads are water-insoluble. In some embodiments, the drug-eluting beads are water-swellable. In some embodiments, the drug-eluting beads are imageable. In some embodiments, the drug-eluting beads are radiopaque. In some embodiments, the drug-eluting beads comprise DC Bead®, HepaSphere®, or CalliSpheres® drug-eluting beads. In some embodiments, the drug-eluting beads comprise DC Bead® drug-eluting beads. In some embodiments, the drug-eluting beads comprise HepaSphere® drug-eluting beads. In some embodiments, the drug-eluting beads comprise CalliSpheres® drug-eluting beads.

In some embodiments, the drug-eluting beads have an average diameter of about 30 μm to about 1200 μm. In some embodiments, the drug-eluting beads have an average diameter of about 70 μm to about 700 μm. In some embodiments, the drug-eluting beads have an average diameter of about 70 μm to about 150 μm, of about 100 μm to about 300 μm, of about 300 μm to about 500 μm, or of about 500 μm to about 700 μm. In some embodiments, the drug-eluting beads have an average diameter of about 30 μm to about 60 μm. In some embodiments, the drug-eluting beads have an average diameter of about 50 μm to about 100 μm. In some embodiments, the drug-eluting beads have an average diameter of about 70 μm to about 150 μm. In some embodiments, the drug-eluting beads have an average diameter of about 100 μm to about 200 μm. In some embodiments, the drug-eluting beads have an average diameter of about 150 μm to about 200 μm. In some embodiments, the drug-eluting beads have an average diameter of about 100 μm to about 300 μm. In some embodiments, the drug-eluting beads have an average diameter of about 300 μm to about 500 μm. In some embodiments, the drug-eluting beads have an average diameter of about 500 μm to about 700 μm. In some embodiments, the drug-eluting beads have an average diameter of about 700 μm to about 900 μm. In some embodiments, the drug-eluting beads have an average diameter of about 900 μm to about 1200 μm.

In some embodiments, the concentration of the drug-eluting beads is from about 0.1 g/mL to about 1 g/mL. In some embodiments, the concentration of the drug-eluting beads is from about 0.1 g/mL to about 0.5 g/mL. In some embodiments, the concentration of the drug-eluting beads is about 0.10 g/mL, about 0.15 g/mL, about 0.20 g/mL, about 0.25 g/mL, about 0.30 g/mL, about 0.35 g/mL, about 0.40 g/mL, about 0.45 g/mL, about 0.50 g/mL, about 0.55 g/mL, about 0.60 g/mL, about 0.65 g/mL, about 0.70 g/mL, about 0.75 g/mL, about 0.80 g/mL, about 0.85 g/mL, about 0.90 g/mL, about 0.95 g/mL, or about 1.0 g/mL. In some embodiments, the concentration of the drug-eluting beads is about 0.25 g/mL.

In some embodiments, the compound is (3S,5R,8R,9S,10S,13R,14S,17R)-14-hydroxy-10,13-dimethyl-17-(2-oxo-2H-pyran-5-yl)hexadecahydro-1H-cyclopenta[α]phenanthren-3-yl piperazine-1-carboxylate (Compound A).

In some embodiments, the aqueous chemoembolization composition comprises from about 0.5 mg to about 5 mg of Compound A per 1 g of drug-eluting beads. In some embodiments, the aqueous chemoembolization composition comprises from about 1 mg to about 2 mg of Compound A per 1 g of drug-eluting beads. In some embodiments, the aqueous chemoembolization composition comprises from about 1.4 mg to about 1.6 mg of Compound A per 1 g of drug-eluting beads. In some embodiments, the chemoembolization composition comprises about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, or about 2.0 mg of Compound A per 1 g of drug-eluting beads. In some embodiments, the aqueous chemoembolization composition comprises about 1.4 mg of Compound A per 1 g of drug-eluting beads. In some embodiments, the aqueous chemoembolization composition comprises about 1.5 mg of Compound A per 1 g of drug-eluting beads. In some embodiments, the aqueous chemoembolization composition comprises about 1.6 mg of Compound A per 1 g of drug-eluting beads.

### II. Methods of Making Chemoembolization Compositions of Compound A

In one aspect, the disclosure provides a method of preparing a chemoembolization composition, the method comprising:
a. providing an aqueous solution of (3S,5R,8R,9S,10S,13R,14S,17R)-14-hydroxy-10,13-dimethyl-17-(2-oxo-2H-pyran-5-yl)hexadecahydro-1H-cyclopenta[α]phenanthren-3-yl piperazine-1-carboxylate (Compound A), or a pharmaceutically acceptable salt thereof; and
b. immersing drug-eluting beads in the aqueous solution of Compound A, wherein Compound A becomes absorbed into the drug-eluting beads.

In some embodiments, the aqueous solution comprises a buffer. In some embodiments, the buffer comprises phosphoric acid, citric acid, acetic acid, histidine, lactic acid, tromethamine, gluconic acid, aspartic acid, glutamic acid, tartaric acid, succinic acid, malic acid, fumaric acid, α-ketoglutaric acid, sodium hydroxide, sodium phosphate, sodium citrate, sodium acetate, potassium hydroxide, potassium phosphate, potassium citrate, potassium acetate, or a combination thereof. In some embodiments, the buffer comprises sodium acetate, acetic acid, or a combination thereof.

In some embodiments, the buffer has a concentration from about 10 mM to about 500 mM. In some embodiments, the buffer has a concentration from about 50 mM to about 200 mM. In some embodiments, the buffer has a concentration of about 10 mM, about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 250 mM, about 300 mM, about 350 mM, about 400 mM, about 450 mM, or about 500 mM. In some embodiments, the buffer has a concentration of about 100 mM.

In some embodiments, the pH of the aqueous solution of Compound A is from about 3.5 to about 7.5. In some embodiments, the pH of the aqueous solution of Compound A is from about 4.5 to about 5.5. In some embodiments, the pH of the aqueous solution of Compound A is about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5. In some embodiments, the pH of the aqueous solution of Compound A is about 5.0.

In some embodiments, the concentration of Compound A in the aqueous solution is from about 0.1 mg/mL to about 1 mg/mL. In some embodiments, the concentration of Compound A in the aqueous solution is from about 0.1 mg/mL to about 0.5 mg/mL. In some embodiments, the concentration of Compound A in the aqueous solution is about 0.10 mg/mL, about 0.15 mg/mL, about 0.20 mg/mL, about 0.25 mg/mL, about 0.30 mg/mL, about 0.35 mg/mL, about 0.40 mg/mL, about 0.45 mg/mL, about 0.50 mg/mL, about 0.55 mg/mL, about 0.60 mg/mL, about 0.65 mg/mL, about 0.70 mg/mL, about 0.75 mg/mL, about 0.80 mg/mL, about 0.85 mg/mL, about 0.90 mg/mL, about 0.95 mg/mL, or about 1.0 mg/mL. In some embodiments, the concentration of Compound A in the aqueous solution is about 0.5 mg/mL.

In some embodiments, the drug-eluting beads are anionic drug-eluting beads. In some embodiments, the drug-eluting beads comprise modified polyvinyl alcohol (PVA) hydrogel beads. In some embodiments, the drug-eluting beads comprise sulfonate-modified polyvinyl alcohol hydrogel beads or carboxyl-modified polyvinyl alcohol acrylate beads. In some embodiments, the drug-eluting beads comprise sulfonate-modified polyvinyl alcohol hydrogel beads. In some embodiments, the drug-eluting beads comprise carboxyl-modified polyvinyl alcohol acrylate beads. In some embodiments, the drug-eluting beads comprise carboxyl-modified polyvinyl alcohol-co-sodium acrylate beads. In some embodiments, the drug-eluting beads are water-insoluble. In some embodiments, the drug-eluting beads are water-swellable. In some embodiments, the drug-eluting beads are imageable. In some embodiments, the drug-eluting beads are radiopaque. In some embodiments, the drug-eluting beads comprise DC Bead®, HepaSphere®, or CalliSpheres® drug-eluting beads. In some embodiments, the drug-eluting beads comprise DC Bead® drug-eluting beads. In some embodiments, the drug-eluting beads comprise HepaSphere® drug-eluting beads. In some embodiments, the drug-eluting beads comprise CalliSpheres® drug-eluting beads.

In some embodiments, the drug-eluting beads have an average diameter of about 30 μm to about 1200 μm. In some embodiments, the drug-eluting beads have an average diameter of about 70 μm to about 700 μm. In some embodiments, the drug-eluting beads have an average diameter of about 70 μm to about 150 μm, of about 100 μm to about 300 μm, of about 300 μm to about 500 μm, or of about 500 μm to about 700 μm. In some embodiments, the drug-eluting beads have an average diameter of about 30 μm to about 60 μm. In some embodiments, the drug-eluting beads have an average diameter of about 50 μm to about 100 μm. In some embodiments, the drug-eluting beads have an average diameter of about 70 μm to about 150 μm. In some embodiments, the drug-eluting beads have an average diameter of about 100 μm to about 200 μm. In some embodiments, the drug-eluting beads have an average diameter of about 150 μm to about 200 μm. In some embodiments, the drug-eluting beads have an average diameter of about 100 μm to about 300 μm. In some embodiments, the drug-eluting beads have an average diameter of about 300 μm to about 500 μm. In some embodiments, the drug-eluting beads have an average diameter of about 500 μm to about 700 μm. In some embodiments, the drug-eluting beads have an average diameter of about 700 μm to about 900 μm. In some embodiments, the drug-eluting beads have an average diameter of about 900 μm to about 1200 μm.

### IV. Methods of Use

In another aspect, the disclosure provides the chemoembolization composition provided herein for use in a method for treating a solid tumor cancer.

In some embodiments, the solid tumor cancer comprises a malignant hypervascularised tumor. In some embodiments, the malignant hypervascularised tumor is selected from the group comprising hepatoma, hepatocellular carcinoma (HCC), liver metastasis, cholangiomas, neuroendocrine tumors, GIST liver metastasis, and renal cancer. In some embodiments, the malignant hypervascularised tumor is hepatoma. In some embodiments, the malignant hypervascularised tumor is hepatocellular carcinoma (HCC). In some embodiments, the malignant hypervascularised tumor is liver metastasis. In some embodiments, the liver metastasis is metastasis from colon cancer. In some embodiments, the liver metastasis is metastasis from breast cancer. In some embodiments, the liver metastasis is metastasis from carcinoid tumors. In some embodiments, the liver metastasis is metastasis from neuroendocrine tumors. In some embodiments, the liver metastasis is metastasis from islet cell tumors of the pancreas. In some embodiments, the liver metastasis is metastasis from ocular melanoma. In some embodiments, the liver metastasis is metastasis from sarcomas. In some embodiments, the liver metastasis is metastasis from a vascular primary tumor. In some embodiments, the malignant hypervascularised tumor is cholangiomas. In some embodiments, the malignant hypervascularised tumor is neuroendocrine tumors. In some embodiments, the malignant hypervascularised tumor is GIST liver metastasis. In some embodiments, the malignant hypervascularised tumor is renal cancer.

In another aspect, the disclosure provides the aqueous chemoembolization composition provided herein for use in a method for treating a solid tumor cancer.

In some embodiments, the solid tumor cancer comprises a malignant hypervascularised tumor. In some embodiments, the malignant hypervascularised tumor is selected from the group comprising hepatoma, hepatocellular carcinoma (HCC), liver metastasis, cholangiomas, neuroendocrine tumors, GIST liver metastasis, and renal cancer. In some embodiments, the malignant hypervascularised tumor is hepatoma. In some embodiments, the malignant hypervascularised tumor is hepatocellular carcinoma (HCC). In some embodiments, the malignant hypervascularised tumor is liver metastasis. In some embodiments, the liver metastasis is metastasis from colon cancer. In some embodiments, the liver metastasis is metastasis from breast cancer. In some embodiments, the liver metastasis is metastasis from carcinoid tumors. In some embodiments, the liver metastasis is metastasis from neuroendocrine tumors. In some embodiments, the liver metastasis is metastasis from islet cell tumors of the pancreas. In some embodiments, the liver metastasis is metastasis from ocular melanoma. In some embodiments, the liver metastasis is metastasis from sarcomas. In some embodiments, the liver metastasis is metastasis from a vascular primary tumor. In some embodiments, the malignant hypervascularised tumor is cholangiomas. In some embodiments, the malignant hypervascularised tumor is neuroendocrine tumors. In some embodiments, the malignant hypervascularised tumor is GIST liver metastasis. In some embodiments, the malignant hypervascularised tumor is renal cancer.

In another aspect, the disclosure provides a method of treating a solid tumor cancer in a subject, the method comprising administering to the subject in need thereof a chemoembolization composition comprising a therapeutically effective amount of (3S,5R,8R,9S,10S,13R,14S,17R)-14-hydroxy-10,13-dimethyl-17-(2-oxo-2H-pyran-5-yl)hexadecahydro-1H-cyclopenta[α]phenanthren-3-yl piperazine-1-carboxylate (Compound A):

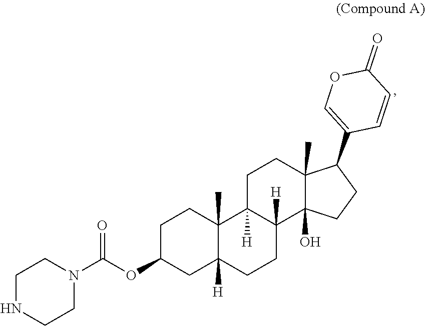

or a pharmaceutically acceptable salt thereof, loaded in drug-eluting beads.

In some embodiments, the solid tumor cancer comprises a malignant hypervascularised tumor. In some embodiments, the malignant hypervascularised tumor is selected from the group comprising hepatoma, hepatocellular carcinoma (HCC), liver metastasis, cholangiomas, neuroendocrine tumors, GIST liver metastasis, and renal cancer. In some embodiments, the malignant hypervascularised tumor is hepatoma. In some embodiments, the malignant hypervascularised tumor is hepatocellular carcinoma (HCC). In some embodiments, the malignant hypervascularised tumor is liver metastasis. In some embodiments, the liver metastasis is metastasis from colon cancer. In some embodiments, the liver metastasis is metastasis from breast cancer. In some embodiments, the liver metastasis is metastasis from carcinoid tumors. In some embodiments, the liver metastasis is metastasis from neuroendocrine tumors. In some embodiments, the liver metastasis is metastasis from islet cell tumors of the pancreas. In some embodiments, the liver metastasis is metastasis from ocular melanoma. In some embodiments, the liver metastasis is metastasis from sarcomas. In some embodiments, the liver metastasis is metastasis from a vascular primary tumor. In some embodiments, the malignant hypervascularised tumor is cholangiomas. In some embodiments, the malignant hypervascularised tumor is neuroendocrine tumors. In some embodiments, the malignant hypervascularised tumor is GIST liver metastasis. In some embodiments, the malignant hypervascularised tumor is renal cancer. In some embodiments, the solid tumor cancer is resectable. In some embodiments, the solid tumor cancer is unresectable.

In another aspect, the disclosure provides a method of treating a hepatocellular carcinoma (HCC) in a subject, the method comprising administering to the subject in need thereof a chemoembolization composition comprising a therapeutically effective amount of (3S,5R,8R,9S,10S,13R,14S,17R)-14-hydroxy-10,13-dimethyl-17-(2-oxo-2H-pyran-5-yl)hexadecahydro-1H-cyclopenta[α]phenanthren-3-yl piperazine-1-carboxylate (Compound A):

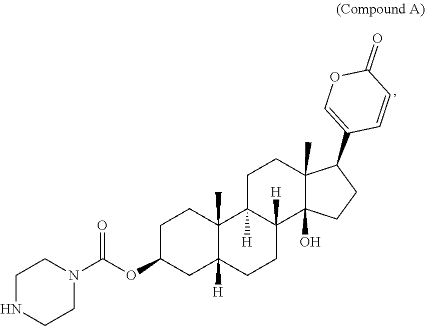

or a pharmaceutically acceptable salt thereof, loaded in drug-eluting beads.

In some embodiments, the HCC is resectable. In some embodiments, the HCC is unresectable.

In some embodiments, the chemoembolization composition is administered directly to the hepatic artery in the liver of the subject. In some embodiments, the chemoembolization composition is administered at a rate of from about 0.25 mL/min to about 2.0 mL/min. In some embodiments, the chemoembolization composition is administered at a rate of about 0.25, about 0.5, about 0.75, about 1.0, about 1.25, about 1.5, about 1.75, or about 2.0 mL/min. In some embodiments, the chemoembolization composition is administered at a rate of about 1.0 mL/min.

In some embodiments, the chemoembolization composition is the chemoembolization composition provided herein. In some embodiments, the chemoembolization composition is the aqueous chemoembolization composition provided herein.

In some embodiments, the subject is treated once with the chemoembolization composition described herein. In some embodiments, the subject is treated more than once with the chemoembolization composition described herein. In some embodiments, the subject is treated one, two, three, or four times in a six-month period with the chemoembolization composition described herein.

In certain embodiments, Compound A is administered in combination with one or more agents chosen from paclitaxel, bortezomib, dacarbazine, gemcitabine, trastuzumab, bevacizumab, capecitabine, docetaxel, erlotinib, aromatase inhibitors, such as AROMASIN™ (exemestane), and estrogen receptor inhibitors, such as FASLODEX™ (fulvestrant).

When Compound A is administered into a human subject, the daily dosage will normally be determined by the prescribing physician with the dosage generally varying according to the age, weight, and response of the individual subject, as well as the severity of the subject's symptoms.

In one exemplary application, a suitable amount of Compound A is administered to a mammal undergoing treatment for cancer. Administration typically occurs in an amount of between about 0.01 mg/kg of body weight to about 100 mg/kg of body weight per day (administered in single or divided doses), such as at least about 0.1 mg/kg of body weight per day. A particular therapeutic dosage can include, e.g., from about 0.01 mg to about 1000 mg of Compound A, such as including, e.g., from about 1 mg to about 1000 mg. The quantity of Compound A in a unit dose of preparation may be varied or adjusted from about 0.1 mg to 1000 mg, such as from about 1 mg to 300 mg, for example 10 mg to 200 mg, according to the particular application. The amount administered will vary depending on the particular IC₅₀ value of Compound A used and the judgment of the attending clinician taking into consideration factors such as health, weight, and age. In combinational applications in which Compound A is not the sole active ingredient, it may be possible to administer lesser amounts of Compound A and still have therapeutic or prophylactic effect.

In some embodiments, the pharmaceutical preparation is in unit dosage form. In such form, the preparation is subdivided into unit doses containing appropriate quantities of Compound A, e.g., an effective amount to achieve the desired purpose.

The actual dosage employed may be varied depending upon the requirements of the subject and the severity of the condition being treated. Determination of the proper dosage for a particular situation is within the skill of the art. Generally, treatment is initiated with smaller dosages which are less than the optimum dose of Compound A. Thereafter, the dosage is increased by small amounts until the optimum effect under the circumstances is reached. For convenience, the total daily dosage may be divided and administered in portions during the day if desired.

The amount and frequency of administration of Compound A, and if applicable other chemotherapeutic agents and/or radiation therapy, will be regulated according to the judgment of the attending clinician (physician) considering such factors as age, condition and size of the subject as well as severity of the disease being treated. In some embodiments, the chemoembolization composition provided herein is administered once every one, two, three, four, five, six, seven, eight, nine, or ten weeks. In some embodiments, the chemoembolization composition provided herein is administered once every four weeks. In some embodiments, the chemoembolization composition provided herein is administered once every eight weeks. In some embodiments, the chemoembolization composition provided herein is administered no more than one, two, three, four, five, six, seven, eight, nine, or ten times in six months. In some embodiments, the chemoembolization composition provided herein is administered no more than four times in six months.

The chemotherapeutic agent and/or radiation therapy can be administered according to therapeutic protocols well known in the art. It will be apparent to those skilled in the art that the administration of the chemotherapeutic agent and/or radiation therapy can be varied depending on the disease being treated and the known effects of the chemotherapeutic agent and/or radiation therapy on that disease. Also, in accordance with the knowledge of the skilled clinician, the therapeutic protocols (e.g., dosage amounts and times of administration) can be varied in view of the observed effects of the administered therapeutic agents (i.e., antineoplastic agent or radiation) on the subject, and in view of the observed responses of the disease to the administered therapeutic agents.

Also, in general, Compound A need not be administered in the same pharmaceutical composition as a chemotherapeutic agent, and may, because of different physical and chemical characteristics, be administered by a different route. For example, Compound A may be administered via TACE, while the chemotherapeutic agent may be administered intravenously. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

The particular choice of chemotherapeutic agent and/or radiation will depend upon the diagnosis of the attending physicians and their judgment of the condition of the subject and the appropriate treatment protocol.

Compound A (and where appropriate chemotherapeutic agent and/or radiation) may be administered concurrently (e.g., simultaneously, essentially simultaneously or within the same treatment protocol) or sequentially, depending upon the nature of the proliferative disease, the condition of the subject, and the actual choice of chemotherapeutic agent and/or radiation to be administered in conjunction (i.e., within a single treatment protocol) with Compound A.

In combinational applications and uses, Compound A and the chemotherapeutic agent and/or radiation need not be administered simultaneously or essentially simultaneously, and the initial order of administration of Compound A and the chemotherapeutic agent and/or radiation, may not be important. Thus, Compound A may be administered first followed by the administration of the chemotherapeutic agent and/or radiation; or the chemotherapeutic agent and/or radiation may be administered first followed by the administration of Compound A. This alternate administration may be repeated during a single treatment protocol. The determination of the order of administration, and the number of repetitions of administration of each therapeutic agent during a treatment protocol, is well within the knowledge of the skilled physician after evaluation of the disease being treated and the condition of the subject. For example, the chemotherapeutic agent and/or radiation may be administered first, and then the treatment continued with the administration of Compound A followed, where determined advantageous, by the administration of the chemotherapeutic agent and/or radiation, and so on until the treatment protocol is complete.

Thus, in accordance with experience and knowledge, the practicing physician can modify each protocol for the administration of Compound A/composition for treatment according to the individual subject's needs, as the treatment proceeds.

The attending clinician, in judging whether treatment is effective at the dosage administered, will consider the general well-being of the subject as well as more definite signs such as relief of disease-related symptoms, inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

### V. Kits/Articles of Manufacture

For use in the therapeutic applications described herein, kits and articles of manufacture are also provided. In some embodiments, such kits comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers are formed from a variety of materials such as glass or plastic.

The articles of manufacture provided herein contain packaging materials. Packaging materials for use in packaging pharmaceutical products include those found in, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. For example, the container(s) includes one or more chemoembolization compositions described herein, optionally in a composition or in combination with another agent as disclosed herein. The container(s) optionally have a sterile access port (for example the container is an intravenous solution bag or a vial having a stopper pierceable by a hypodermic injection needle). Such kits optionally comprising a compound with an identifying description or label or instructions relating to its use in the methods described herein.

For example, a kit typically includes one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included. A label is optionally on or associated with the container. For example, a label is on a container when letters, numbers or other characters forming the label are attached, molded or etched into the container itself, a label is associated with a container when it is present within a receptacle or carrier that also holds the container, e.g., as a package insert. In addition, a label is used to indicate that the contents are to be used for a specific therapeutic application. In addition, the label indicates directions for use of the contents, such as in the methods described herein. In certain embodiments, the pharmaceutical composition is presented in a pack or dispenser device which contains one or more unit dosage forms containing a compound provided herein. The pack for example contains metal or plastic foil, such as a blister pack. Or, the pack or dispenser device is accompanied by instructions for administration. Or, the pack or dispenser is accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In some embodiments, chemoembolization compositions containing Compound A formulated in a compatible pharmaceutical carrier are prepared, placed in an appropriate container, and labeled for treatment of an indicated condition.

### EXAMPLES

### Example 1. Compound a Loading onto Microspheres

Microspheres (1 g, 100-300 μm, CalliSpheres® CallisynBiomedical, Inc.) were immersed into Compound A solution (2 mg in 4 ml of NaOAc/HOAc buffer (100 mM, pH5.0)) using a 10 mL disposable syringe. Residual compound concentration remaining in the depleted loading solution over time was determined by HPLC method.

TABLE 1
Concentration of residual compound and amount
of compound loaded onto microspheres

| | Concentration | Amount of |
|---|---|---|
| Time | of Residual | Compound Loaded |
| (h) | (mg/mL) | (mg) |
| 0 | 0.458 | 0 |
| 0.25 | 0.091 | 1.47 |
| 0.5 | 0.081 | 1.51 |
| 0.75 | 0.083 | 1.50 |
| 1 | 0.060 | 1.59 |

### Example 2. Elution of Compound a Using the T-Apparatus

The microspheres loaded with Compound A were eluted into 50 mL of phosphate buffer solution (PBS, pH 7.4) at 25° C. using a T-Apparatus (Lewis A L, Gonzalez M V, Lloyd A W, et al. DC bead: in vitro characterization of a drug-delivery device for transarterial chemoembolization. J Vasc Interv Radiol, 2006, 17: 335-42). The concentration of Compound A was determined using HPLC method.

TABLE 2
Concentration of compound eluted

| | Concentration of | Amount of |
|---|---|---|
| Time | Compound Eluted | Compound Eluted |
| (h) | (mg/mL) | (mg) |
| 0.083 | 0.013 | 0.63 |
| 0.5 | 0.014 | 0.67 |
| 0.75 | 0.014 | 0.70 |
| 1 | 0.014 | 0.70 |
| 2 | 0.015 | 0.76 |
| 4 | 0.019 | 0.94 |
| 6 | 0.022 | 1.10 |
| 24 | 0.024 | 1.21 |

HPLC was performed using the following instrument and/or conditions:

| Column: | Agilent ZORBAX, SB-Aq, 150 × 4.6 mm, 5 μm | | |
|---|---|---|---|
| Column temperature: | 25° C. | | |
| Detection: | 300 nm | | |
| Injection volume: | 20 μL | | |
| Flow rate: | 1.0 mL/min | | |
| Run time: | 15 min | | |
| Mobile Phase A: | Acetonitrile | | |
| Mobile Phase B: | 0.1% Trifluoroacetic acid | | |
| | Time | Mobile | Mobile |
| | (minutes) | Phase A (%) | Phase B (%) |
| Gradient | 0 | 15 | 85 |
| | 13 | 90 | 10 |
| | 15 | 90 | 10 |

### Example 3. Single Arm Study of Safety and Efficacy of Compound a Delivered Via Drug Eluting Beads Transarterial Chemoembolization (DEB-TACE) in Patients with Hepatocellular Carcinoma

To investigate the safety and efficacy of Compound A delivered via DEB-TACE in patients with unresectable hepatocellular carcinoma (HCC), a phase II, open-label, single arm study was conducted.

Compound A is loaded onto CalliSpheres® beads. Briefly, saline solution was removed from CalliSpheres® beads, and Compound A solution (4 mL, 0.5 mg/mL in NaOAc/HOAc buffer (100 mM, pH5.0) is added and left for 15 min. Loaded beads are aspirated into a syringe and nonionic contrast medium is added. After the tumor is confirmed by angiography and stained, loaded beads are slowly delivered at an injection rate of 1 mL/min. Tumors are evaluated via CT/MIRI imaging after 4 weeks.

If the results show complete tumor tissue necrosis, no increase for active lesions, and no new lesions per mRECIST criteria, then no further TACE treatment is needed; otherwise, patients need to undergo further TACE treatments based on the judgement of principal investigators. TACE treatment is conducted less than 4 times during six months period. After three months of last TACE treatment, the efficacy is evaluated every 8 weeks. All subjects are carefully monitored every 4 or 8 weeks followed for adverse events (AEs) during the study treatment. AEs are graded according to the National Cancer Institute Common Terminology Criteria for Adverse Events (NCI CTCAE), Version 5.0.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A chemoembolization composition comprising (3S,5R,8R,9S,10S,13R,14S,17R)-14-hydroxy-10,13-dimethyl-17-(2-oxo-2H-pyran-5-yl)hexadecahydro-1H-cyclopenta[α]phenanthren-3-yl piperazine-1-carboxylate (Compound A): 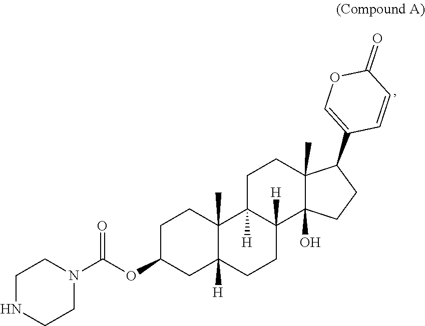
or a pharmaceutically acceptable salt thereof, loaded in drug-eluting beads.

2. The chemoembolization composition of claim 1, wherein the drug-eluting beads are anionic drug-eluting beads.

3. The chemoembolization composition of claim 1 or claim 2, wherein the drug-eluting beads comprise modified polyvinyl alcohol (PVA) hydrogel beads.

4. The chemoembolization composition of claim 1 or claim 2, wherein the drug-eluting beads comprise sulfonate-modified polyvinyl alcohol hydrogel beads or carboxyl-modified polyvinyl alcohol acrylate beads.

5. The chemoembolization composition of claim 1, wherein the drug-eluting beads comprise DC Bead®, HepaSphere®, or CalliSpheres® drug-eluting beads.

6. The chemoembolization composition of any one of claims 1-5, wherein the drug-eluting beads have an average diameter of about 70 μm to about 700 μm.

7. The chemoembolization composition of any one of claims 1-6, wherein the drug-eluting beads have an average diameter of about 70 μm to about 150 μm, of about 100 μm to about 300 μm, of about 300 μm to about 500 μm, or of about 500 μm to about 700 μm.

8. The chemoembolization composition of any one of claims 1-7, wherein the drug-eluting beads have an average diameter of about 100 μm to about 300 μm.

9. The chemoembolization composition of any one of claims 1-8, wherein the compound is (3S,5R,8R,9S,10S,13R,14S,17R)-14-hydroxy-10,13-dimethyl-17-(2-oxo-2H-pyran-5-yl)hexadecahydro-1H-cyclopenta[α]phenanthren-3-yl piperazine-1-carboxylate (Compound A).

10. The chemoembolization composition of any one of claims 1-9, comprising from about 0.5 mg to about 5 mg of Compound A per 1 g of drug-eluting beads.

11. The chemoembolization composition of any one of claims 1-9, comprising from about 1 mg to about 2 mg of Compound A per 1 g of drug-eluting beads.

12. The chemoembolization composition of any one of claims 1-9, comprising from about 1.4 mg to about 1.6 mg of Compound A per 1 g of drug-eluting beads.

13. The chemoembolization composition of any one of claims 1-12, for use in a method for treating a solid tumor cancer.

14. The chemoembolization composition of claim 13, wherein the solid tumor cancer comprises a malignant hypervascularised tumor.

15. The chemoembolization composition of claim 14, wherein the malignant hypervascularised tumor is selected from the group comprising hepatoma, hepatocellular carcinoma (HCC), liver metastasis, cholangiomas, neuroendocrine tumors, GIST liver metastasis, and renal cancer.

16. An aqueous chemoembolization composition comprising (3S,5R,8R,9S,10S,13R,14S,17R)-14-hydroxy-10,13-dimethyl-17-(2-oxo-2H-pyran-5-yl)hexadecahydro-1H-cyclopenta[α]phenanthren-3-yl piperazine-1-carboxylate (Compound A): 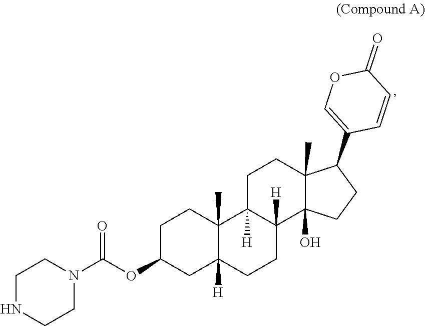
or a pharmaceutically acceptable salt thereof, loaded in drug-eluting beads.

17. The aqueous chemoembolization composition of claim 16, further comprising a buffer.

18. The aqueous chemoembolization composition of claim 17, wherein the buffer comprises phosphoric acid, citric acid, acetic acid, histidine, lactic acid, tromethamine, gluconic acid, aspartic acid, glutamic acid, tartaric acid, succinic acid, malic acid, fumaric acid, α-ketoglutaric acid, sodium hydroxide, sodium phosphate, sodium citrate, sodium acetate, potassium hydroxide, potassium phosphate, potassium citrate, potassium acetate, or a combination thereof.

19. The aqueous chemoembolization composition of claim 17 or claim 18, wherein the buffer comprises sodium acetate, acetic acid, or a combination thereof.

20. The aqueous chemoembolization composition of any one of claims 17-19, wherein the buffer has a concentration from about 10 mM to about 500 mM.

21. The aqueous chemoembolization composition of any one of claims 17-20, wherein the buffer has a concentration of about 100 mM.

22. The aqueous chemoembolization composition of any one of claims 17-21, wherein the pH of the aqueous chemoembolization composition is from about 3.5 to about 7.5.

23. The aqueous chemoembolization composition of any one of claims 17-22, wherein the pH of the aqueous chemoembolization composition is from about 4.5 to about 5.5.

24. The aqueous chemoembolization composition of any one of claims 17-23, wherein the pH of the aqueous chemoembolization composition is about 5.0.

25. The aqueous chemoembolization composition of any one of claims 16-24, wherein the drug-eluting beads are anionic drug-eluting beads.

26. The aqueous chemoembolization composition of any one of claims 16-25, wherein the drug-eluting beads comprise modified polyvinyl alcohol (PVA) hydrogel beads.

27. The aqueous chemoembolization composition of any one of claims 16-26, wherein the drug-eluting beads comprise sulfonate-modified polyvinyl alcohol hydrogel beads or carboxyl-modified polyvinyl alcohol acrylate beads.

28. The aqueous chemoembolization composition of any one of claims 16-27, wherein the drug-eluting beads comprise DC Bead®, HepaSphere®, or CalliSpheres® drug-eluting beads.

29. The aqueous chemoembolization composition of any one of claims 16-28, wherein the drug-eluting beads have an average diameter of about 70 μm to about 700 μm.

30. The aqueous chemoembolization composition of any one of claims 16-29, wherein the drug-eluting beads have an average diameter of about 70 μm to about 150 μm, of about 100 μm to about 300 μm, of about 300 μm to about 500 μm, or of about 500 μm to about 700 μm.

31. The aqueous chemoembolization composition of any one of claims 16-30, wherein the drug-eluting beads have an average diameter of about 100 μm to about 300 μm.

32. The aqueous chemoembolization composition of any one of claims 16-31, wherein the concentration of the drug-eluting beads is from about 0.1 g/mL to about 1 g/mL.

33. The aqueous chemoembolization composition of any one of claims 16-32, wherein the concentration of the drug-eluting beads is from about 0.1 g/mL to about 0.5 g/mL.

34. The aqueous chemoembolization composition of any one of claims 16-33, wherein the concentration of the drug-eluting beads is about 0.25 g/mL.

35. The aqueous chemoembolization composition of any one of claims 16-34, wherein the compound is (3S,5R,8R,9S,10S,13R,14S,17R)-14-hydroxy-10,13-dimethyl-17-(2-oxo-2H-pyran-5-yl)hexadecahydro-1H-cyclopenta[α]phenanthren-3-yl piperazine-1-carboxylate (Compound A).

36. The aqueous chemoembolization composition of any one of claims 16-35, comprising from about 0.5 mg to about 5 mg of Compound A per 1 g of drug-eluting beads.

37. The aqueous chemoembolization composition of any one of claims 16-36, comprising from about 1 mg to about 2 mg of Compound A per 1 g of drug-eluting beads.

38. The aqueous chemoembolization composition of any one of claims 16-37, comprising from about 1.4 mg to about 1.6 mg of Compound A per 1 g of drug-eluting beads.

39. The aqueous chemoembolization composition of any one of claims 16-38, for use in a method for treating a solid tumor cancer.

40. The aqueous chemoembolization composition of claim 39, wherein the solid tumor cancer comprises a malignant hypervascularised tumor.

41. The aqueous chemoembolization composition of claim 40, wherein the malignant hypervascularised tumor is selected from the group comprising hepatoma, hepatocellular carcinoma (HCC), liver metastasis, cholangiomas, neuroendocrine tumors, GIST liver metastasis, and renal cancer.

42. A method of treating a solid tumor cancer in a subject, the method comprising administering to the subject in need thereof a chemoembolization composition comprising a therapeutically effective amount of (3S,5R,8R,9S,10S,13R,14S,17R)-14-hydroxy-10,13-dimethyl-17-(2-oxo-2H-pyran-5-yl)hexadecahydro-1H-cyclopenta[α]phenanthren-3-yl piperazine-1-carboxylate (Compound A): 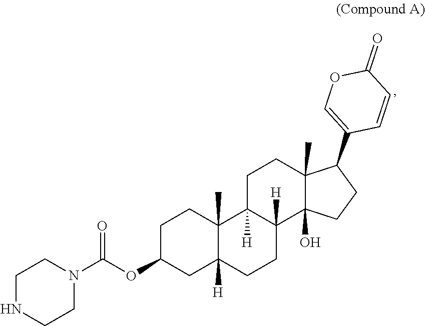
or a pharmaceutically acceptable salt thereof, loaded in drug-eluting beads.

43. The method of claim 42, wherein the solid tumor cancer comprises a malignant hypervascularised tumor.

44. The method of claim 43, wherein the malignant hypervascularised tumor is selected from the group comprising hepatoma, hepatocellular carcinoma (HCC), liver metastasis, cholangiomas, neuroendocrine tumors, GIST liver metastasis, and renal cancer.

45. The method of any one of claims 42-44, wherein the solid tumor cancer is unresectable.

46. A method of treating a hepatocellular carcinoma (HCC) in a subject, the method comprising administering to the subject in need thereof a chemoembolization composition comprising a therapeutically effective amount of (3S,5R,8R,9S,10S,13R,14S,17R)-14-hydroxy-10,13-dimethyl-17-(2-oxo-2H-pyran-5-yl)hexadecahydro-1H-cyclopenta[α]phenanthren-3-yl piperazine-1-carboxylate (Compound A): 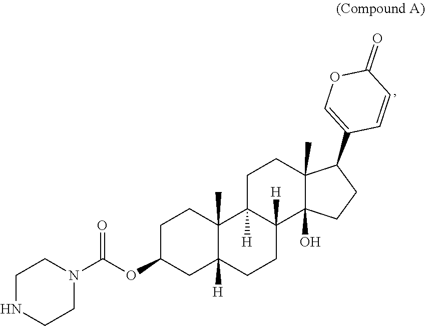
or a pharmaceutically acceptable salt thereof, loaded in drug-eluting beads.

47. The method of claim 46, wherein the HCC is unresectable.

48. The method of claim 46 or claim 47, wherein the chemoembolization composition is administered directly to the hepatic artery in the liver of the subject.

49. The method of any one of claims 42-48, wherein the chemoembolization composition comprises from about 1.4 mg to about 1.6 mg of Compound A and about 1 g of drug-eluting beads.

50. The method of any one of claims 42-49, wherein the chemoembolization composition is the chemoembolization composition of any one of claims 1-12.

51. The method of any one of claims 42-49, wherein the chemoembolization composition is the aqueous chemoembolization composition of any one of claims 16-38.
